(19) United States
(12) Patent Application Publication
(10) Pub. No.: US 2025/0268584 A1
(43) Pub. Date: Aug. 28, 2025

(54) PORTABLE URINE STORAGE DEVICE WITH PROGRAMMABLE TEMPERATURE AND SIMULATION FUNCTIONALITY

(71) Applicant: Bryan Keith Sullivan, Largo, FL (US)

(72) Inventor: Bryan Keith Sullivan, Largo, FL (US)

(21) Appl. No.: 19/206,728

(22) Filed: May 13, 2025

Related U.S. Application Data
(62) Division of application No. 15/929,265, filed on Apr. 20, 2020, now abandoned.
(60) Provisional application No. 62/836,436, filed on Apr. 19, 2019.

(51) Int. Cl. A61B 10/00; B01L 3/00; B01L 7/00

(52) U.S. Cl. CPC A61B 10/007; B01L 3/502; B01L 7/00; A61B 2560/0431; B01L 2200/18; B01L 2300/042; B01L 2300/06; B01L 2300/1805

(57) ABSTRACT

A method of simulating urination of temperature-regulated stream of urine through a portable urine storage device is provided. The urine storage device for maintaining urine at selectable and controllable temperatures during transportation. The fluid storage device nests a container in a heating element. A temperature probe is in the container and electrically coupled to a programmable thermostat powering the heating element. The urine storage device enables the current temperature and a desired temperature to be represented on a user interface along the housing of the container. A release switch valve enables the release of the urine from the container in a manner that simulates human urination in form and temperature.

### CROSS-REFERENCE TO RELATED APPLICATION

This application claims the benefit of priority of U.S. provisional application No. 62/836,436, filed 19 Apr. 2019, the contents of which are herein incorporated by reference.

This application also claims the benefit of priority of U.S. Non-provisional application Ser. No. 15/929,265, filed 20 Apr. 2020, the contents of which are herein incorporated by reference.

### BACKGROUND OF THE INVENTION

The present invention relates to fluid containers and, more particularly, to a portable fluid storage device with electronically controlled temperature and digital functionality. The fluid could be urine or other liquids, depending on the preferences and needs of the user.

Urine samples are difficult to maintain at human body temperature. Some other products use hand warmers to maintain urine temperature. Others use 9-volt batteries. Yet others require the use of syringes and microwaving the urine before use. All have unreliable temperature control, no digital temperature display, and no user input for selecting a desired urine temperature.

As can be seen, there is a need for a fluid storage device with electronically controlled temperature and digital functionality. The device embodied in the present invention includes a container for retaining a fluid such as urine. The container is housed in a housing also providing a power source that warms urine very quickly, does not require microwaving or syringes, and has user interface enabling a digital temperature display and programmable temperature controls for electronically selecting a desired urine temperature. The housing for the container also provides a release switch valve that when engaged urges the urine to flow from the container to simulate natural human urination.

### SUMMARY OF THE INVENTION

In one aspect of the present invention, a temperature-regulated urine storage device includes the following: a housing having a user interface along a housing exterior; a heating element having a hollow; a container providing a cavity; the container nested in the hollow; a spout fluidly coupled to the cavity and an external portion of the container; a thermal probe in the cavity; and a thermostat electrically coupled to the thermal probe, the heating element, and the user interface in such a way that a fluid temperature sensed by the thermal probe is representable on the user interface and a heating temperature is settable through the user interface.

In another aspect of the present invention, the temperature-regulated urine storage device includes the following: a power switch electrically coupled to the thermostat; a sealant between the container and the thermostat, the sealant generally overlapping a bottom portion of the heating element; a sheath protecting the thermal probe in the cavity; a cap engaging the spout; the cap movably between an open condition and a closed condition preventing flow from the cavity; a release switch valve having an external component along the housing exterior; and the release switch valve having an internal component coupled to the container in such a way that urging a fluid from the cavity simulates human urination in form and temperature, wherein the fluid is urine and the cavity is dimensioned to store two to three fluid ounces of urine; and a pouch with a strap, the pouch dimensioned to snugly receive the housing.

In yet another aspect of the present invention, a urine storage system includes the following: the above-mentioned temperature-regulated urine storage device; the housing providing a container compartment for the heating element and the sealant; an electronics compartment disposed below the container compartment; the electronics compartment housing the thermostat; and a separator plate between the container compartment and the electronics compartment.

In yet another aspect of the present invention, a method of effectuating a urine fetish simulating a stream of temperature-appropriate urine provides the following: providing the above-mentioned temperature-regulated urine storage device; moving the cap to the open condition; and activating the external component of the release switch valve. And then strapping the strap to a movable object for transportation of said storage device to another location.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is a front elevation view of an exemplary embodiment of the present invention;

FIG. 3 is a front elevation view of an exemplary embodiment of the present invention; and

FIG. 4 is a flow chart of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a portable fluid storage device for maintaining urine at selectable and controllable temperatures during transportation. The fluid storage device nests a container in a heating element. A temperature probe is in the container and electrically coupled to a programmable thermostat powering the heating element. As a result, the current temperature and a desired temperature can be represented on a user interface along a housing of the container. A release switch valve enables the release of the urine from the container in a manner that simulates human urination in form and temperature.

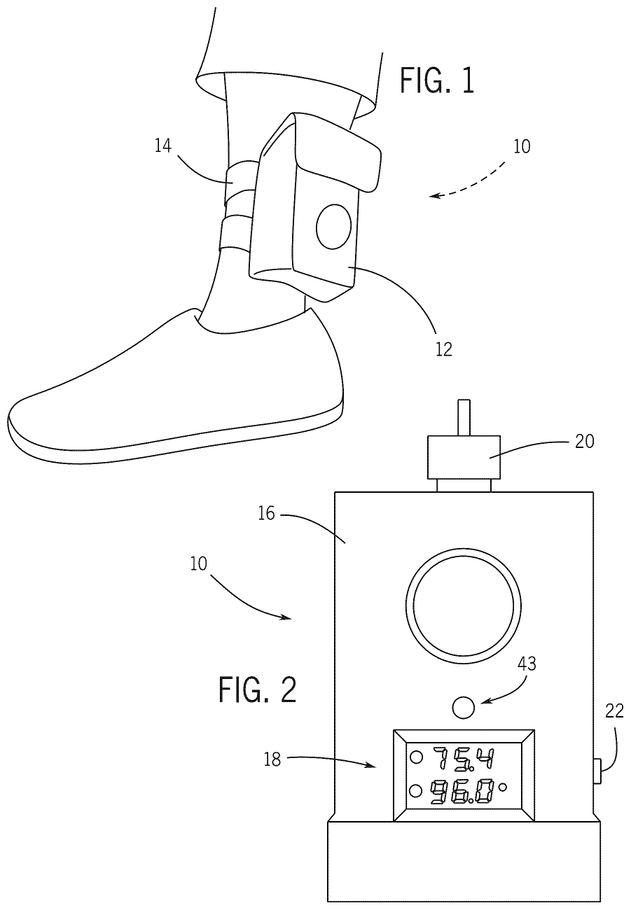

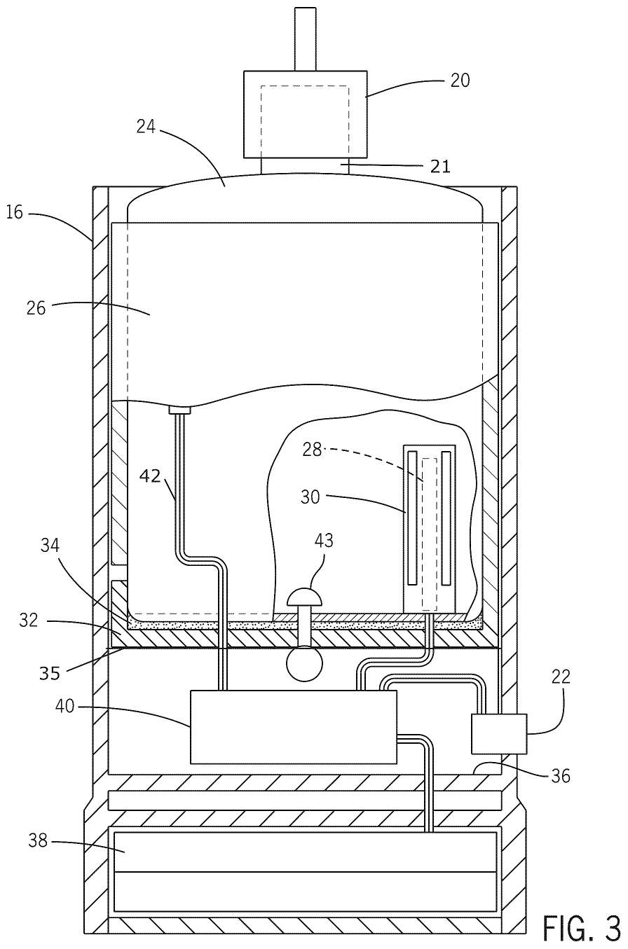

Referring to FIGS. 1 through 4, the present invention may include a portable fluid storage device 10. The portable fluid storage device 10 may include a housing 16 providing an externally visible user interface 18. The user interface may include a digital display and a control unit through which a user can input and program desired output, including temperature related readings and control. The housing 16 defines a compartment for storing a container 24, as illustrated in FIG. 3. A spout 21 on the container 24 fluidly connects to the cavity for filling or dispensing fluid in or out of the cavity. The cap 20 may engage the spout 21 to fluidly seal off the spout 21.

A heating element 26, such as a warming pad wrap, may provide a hollow for receiving the container 24 in a nested condition. The nested condition can be received within the compartment, as illustrated in FIG. 3. A sealant 34 may interconnect the bottom of the container 24 and/or heating element 26 with the housing or a bottom layer thereof. The sealant 34 may be silicone adhesive or like.

In another space or compartment in the housing 16, a power source 38 may be provided. The power source 38 may be rechargeable batteries, such as two 14500 size batteries, lithium ion 850 MAH. Engaging the container 24, either on the inside or the outside thereof, may be a temperature probe 28. The temperature probe 28 may be sheathed in a protective sheath 30. In one embodiment, where in the temperature probe 28 is in the container, a hole is in a bottom layer 32 of the container 24 and or the compartment for associated electrical wiring to connect to the power source 38 and/or a thermostat.

The housing 16 may house a programmable digital thermostat 40. The programmable digital thermostat 40 may provide insulated wires 42 for electrically connecting to the heating element 26 and the temperature probe 28 for sensing and controlling power thereto and thus its heat generated. The programmable digital thermostat 40 and temperature probe 28 may be electrically connected to the power supply 38 and a power switch 22 disposed on an exterior of the housing 16. A first separator plate 35 may be disposed between the fluid container 24 and the power source 38, the thermostat 40, and power switch 38. A second separator plate 36 may be disposed between the power source 38 and the thermostat 40 and power switch 38.

Disposed in the container 24 and along an exterior of the housing 16 may be a release switch valve 43. Engaging the release switch valve 43 along the exterior of the housing 16 releases the urine flow from the container 24 to simulate natural human urination. In certain embodiments, the simulation may include flipping the cap 20 and depressing the externally provided component of the release switch valve 43 (as illustrated in FIG. 2) to release the fluid from the spout 21.

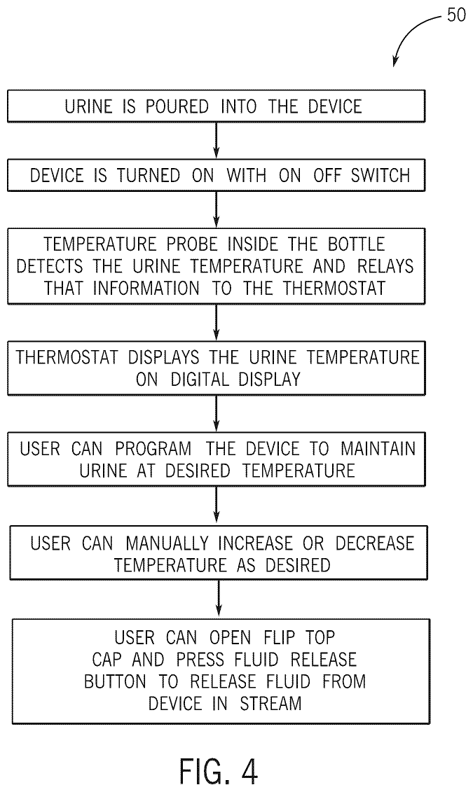

Referring to FIG. 4, and flowchart 50, a method of using the present invention may include the following. The fluid storage device 10 disclosed above may be provided. A user may pour urine into the cavity through the spout 21. The fluid storage device 10 is turned on through the user engaging the power switch 22. The temperature probe 28 inside the container 24 detects the urine's temperature and relays that information to the programmable digital thermostat 40. The thermostat 40 displays the urine temperature on the user interface 18 digital display. The thermostat 40 is user programmable through the user interface 18 for selectively setting the urine temperature. The power source 38 enables the heating element 26 to heat the urine inside container 24 to the temperature programmed selected by user. The thermostat 40 enables the user to maintain the urine's temperature as desired. The user can choose digital display in Celsius or Fahrenheit by pressing a C/F button on thermostat. If the temperature is too low, user can increase or decrease the temperature on the thermostat via, in certain embodiments, a “SET” button. The present invention can be used for novelty or fetish purposes. The container 24 is dimensioned to store one to twelve ounces of fluid. The fluid storage device 10 may be carried in a pouch 12 with a strap 12 than can be removably attached to an object or part of a user's body.

It should be understood, of course, that the foregoing relates to exemplary embodiments of the invention and that modifications may be made without departing from the spirit and scope of the invention as set forth in the following claims.

What is claimed is:

1. A method of effectuating a urine fetish simulating a stream of temperature-appropriate urine, comprising:
flipping a flip cap of a temperature-regulated urine storage device to an open condition, the temperature-regulated urine storage device comprises:
a housing having a user interface along a housing exterior;
a heating element having a hollow;
a container providing a cavity; the container nested in the hollow;
a spout fluidly coupled to the cavity and an external portion of the container;
the flip cap movable between the open condition and a closed condition;
a thermal probe in the cavity;
a thermostat electrically coupled to the thermal probe, the heating element, and the user interface in such a way that a fluid temperature sensed by the thermal probe is representable on the user interface and a heating temperature is settable through the user interface;
the housing has one main compartment wherein a separator plate divides the main compartment into a container compartment housing the container and an electronics compartment housing the thermostat;
a release switch valve having an external component along an exterior of the housing;
the release switch valve movable between an enabled stated and a disenabled state;
the release switch valve having an internal component extending through the housing and into the container in such a way to, when the release switch valve is in the enabled state, a fluid is urge through and out of the spout as a stream to simulate human urination in form and temperature; and
activating the external component to move the release switch valve to the enabled state.

2. The method of claim 1, further comprising:
strapping the strap to a movable object for transportation of said storage device to an ankle area of a human wearer.
